(19) United States
(12) Patent Application Publication
(10) Pub. No.: US 2024/0138449 A1
(43) Pub. Date: May 2, 2024

(54) Method for Preparing Moisture-Proof Amino Acid Materials

(71) Applicant: NANJING NUTRABUILDING BIO-TECH CO., LTD., Nanjing (CN)

(72) Inventors: Jun Tang, Nanjing (CN); Wei Wang, Nanjing (CN); Ou Wang, Nanjing (CN); Qiru Fan, Nanjing (CN); Kylin Liao, Nanjing (CN)

(73) Assignee: NANJING NUTRABUILDING BIO-TECH CO., LTD., Nanjing (CN)

(21) Appl. No.: 17/771,558

(22) PCT Filed: Mar. 17, 2022
(86) PCT No.: PCT/CN2022/081353

§ 371 Date: May 11, 2023

(30) Foreign Application Priority Data
Nov. 4, 2021 (WO) PCT/CN2021/128653

(51) Int. Cl. A23L 3/46; A23L 33/175

(52) U.S. Cl. CPC A23L 3/46; A23L 33/175

(57) ABSTRACT

The present invention provides novel methods for preparing a moisture-proof amino acid material. The present invention also provides novel compositions or nutritional supplements comprising the moisture-proof amino acid materials prepared by the methods of this invention.

### CROSS-REFERENCE TO RELATED APPLICATION

This application claims priority to International Application No. PCT/CN2021/128653, filed on Nov. 4, 2021, the contents of which are incorporated herein by reference in their entirety.

### BACKGROUND OF THE INVENTION

Water-soluble amino acids such as alanine, aminoisobutyric acid, phenylalanine, tryptophan, methionine, valine, proline, glycine, serine, threonine, cysteine, aspartic acid, lysine, arginine, histidine, glutamic acid, etc. are prone to moisture absorption in the air. Absorbed moisture causes poor weighing and feeding of these amino acids in the production of products containing these amino acids. The moisture also results in local stickiness in the products containing these amino acids. These effects have a negative impact on the storage and production process of products containing these amino acids, and also affects the quality and stability of these products.

To solve the problem of moisture absorption of these amino acids, the common method used in the industry is to encapsulate these amino acids through a granulation coating process to isolate them from the external environment, so as to achieve the purpose of preventing moisture absorption. However, this preparation process is cumbersome, long cycle time, high energy consumption, not economic and environmentally friendly.

Another common practice is through the addition of anti-caking agent, but its moisture-proof effect is not significant. In addition, anti-caking agent is a food additive, the use of which subjects to strict regulations. Moreover, addition of anti-caking agent to some products, such as nutritional supplements, will also affect the taste of the product.

Therefore, there is a need to develop a simple, feasible and moisture-proof treatment method for amino acids at present stage.

### SUMMARY OF THE INVENTION

In one aspect, the present invention provides a method of preparing moisture-proof amino acid materials. The method includes the following steps 1) preparing a first solution or suspension of an amino acid by mixing the amino acid in a dispersion medium, 2) mixing a moisture-proof excipient with the first solution or suspension to form a second solution or suspension, and 3) drying the second solution or suspension to remove the dispersion medium to obtain a moisture-proof amino acid material.

In some embodiments, the amino acid comprises β-aminoisobutyric acid or β-alanine.

In some embodiments, the amino acid comprises one or more amino acids selected from the group consisting of alanine, aminoisobutyric acid, phenylalanine, tryptophan, methionine, valine, proline, glycine, serine, threonine, cysteine, aspartic acid, lysine, arginine, histidine, and glutamate.

In some embodiments, the moisture-proof excipient accounts for 5% to 20% of the total weight of the moisture-proof excipient and the amino acid.

In some embodiments, the moisture-proof excipient comprises leucine.

In some embodiments, the moisture-proof excipient is added into the first solution or suspension that contains the amino acid. In some embodiments, the moisture-proof excipient is firstly dispersed in a dispersion medium, then mixed with the first solution or suspension to form the second solution or suspension.

In some embodiments, the dispersion medium comprises water.

In some embodiments, the drying process includes spray drying, reduced pressure drying, freeze drying, or microwave drying. In some embodiments, the drying process includes spray drying.

In some embodiments, the drying process includes two or more of the following drying methods: spray drying, reduced pressure drying, freeze drying, and microwave drying.

In some embodiments, a moisture-proof amino acid material can also be used as a nutritional supplement to provide enriched amino acids.

Some embodiments relate to a composition comprising the moisture-proof amino acid material prepared from any methods disclosed herein.

Some embodiments relate to a nutritional supplement comprising the moisture-proof amino acid material obtained herein.

These and other features, aspects, and advantages of the present invention will become better understood with reference to the following description and appended claims.

### DETAILED DESCRIPTION OF THE INVENTION

In the Summary Section above and the Detailed Description Section, and the claims below, reference is made to particular features of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

The present invention provides novel methods for preparing a moisture-proof amino acid material. In some embodiments, the method comprises the following steps: 1) preparing a first solution or suspension of an amino acid by mixing the amino acid in a dispersion medium, 2) mixing a moisture-proof excipient with the first solution or suspension to form a second solution or suspension, and 3) drying the second solution or suspension to remove the dispersion medium to obtain a moisture-proof amino acid material. Some moisture-proof amino acid material scan also be used as a nutritional supplement to provide enriched amino acids.

Here, the use of “an amino acid” does not exclude a plurality. “An amino acid” could be one amino acid or a mixture of multiple amino acids.

“Material” or “materials” used herein include all items related to the production of products, such as raw materials, auxiliary supplies, semi-finished products, etc. An “amino acid material” refers to a material contains one or more types of amino acid as components. The amino acid materials prepared according to methods disclosed herein can be directly used as a dietary or nutritional supplement for the end consumers. Or the amino acid materials can be used as an intermediate ingredient for those dietary or nutritional supplements. The amino acid materials disclosed herein can also be used in other products, such as animal food or food additives, where amino acids are usually added.

As used herein, “dispersion medium” can refer to a solvent, in which substances (or solutes) are dissolved forming a solution. In a solution, the solvent is the one that is usually present in greater amount than the solute. “Dispersion medium” can also refer to a medium in which particles are dispersed but not dissolved in such a medium.

In some embodiments, the amino acid is β-aminoisobutyric acid or β-alanine. β-aminoisobutyric acid (3-amino-2-methylpropanoic acid or BAIBA), of the molecular formula C₄H₉N₂ is easily soluble in water. It can be used to improve abnormal lipid metabolism and liver endoplasmic reticulum stress in type 2 diabetes and is a beneficial amino acid for human body. β-Alanine, a colorless crystal of the molecular formula C₃H₇NO₂, is readily soluble in water. It is the basic unit of protein, and is one of the 20 amino acids that make up human protein, and can be used to prevent kidney stones, assist in the metabolism of glucose, help ease hypoglycemia, and improve body energy, etc.

In some embodiments, the amino acid includes one or more amino acids selected from the group consisting of alanine, aminoisobutyric acid, phenylalanine, tryptophan, methionine, valine, proline, glycine, serine, threonine, cysteine, aspartic acid, lysine, arginine, histidine, and glutamate.

Some of the amino acids listed herein are beneficial to human being and can be added to a nutritional supplement. However, these amino acids are prone to absorbing moisture in the air. Absorbed moisture causes poor weighing and feeding of these amino acids in the production of nutritional supplements containing these amino acids. The moisture also results in local stickiness in the nutritional supplements containing these amino acids. These effects have a negative impact on the storage and production process of nutritional supplements containing these amino acids, and also affects the quality and stability of these nutritional supplements. Some of such issues can be mitigated by using the methods disclosed herein to mix these amino acids with moisture-proof excipients.

In some embodiments, the moisture-proof excipient accounts for 1% to 99% or 1% to 30% (by weight) of the total weight of the moisture-proof excipient and the amino acid. The recitation of numerical ranges, as used herein, is intended to be inclusive and such numerical ranges should be construed as covering any number in between. For example, 1 through 10 should be considered to cover 2 to 8, 3 to 7, 5 to 6, 1 to 9, 3.6 to 4.6, 3.5 to 9.9, and the like. In some embodiments, the moisture-proof excipient accounts for approximately 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 percent of the total weight of the moisture-proof excipient and the amino acid. In some embodiments, the moisture-proof excipient accounts for 5% to 20% of the total weight of the moisture-proof excipient and the amino acid.

As used herein, an “excipient” refers to an inert substance that is added to a composition to provide, without limitation, bulk, consistency, stability, binding ability, lubrication, disintegrating ability etc., to the composition. An “excipient” is an auxiliary material in a composition.

In some embodiments, the moisture-proof excipient is leucine. Leucine is one of nine essential amino acids in humans (provided by food), and it is important for protein synthesis and many metabolic functions. Leucine is also a functional excipient. However, overdose of leucine can be toxic. Leucine toxicity, as seen in decompensated maple syrup urine disease, causes delirium and neurologic compromise, and can be life-threatening. A high intake of leucine may cause or exacerbate symptoms of pellagra in people with low niacin status because it interferes with the conversion of L-tryptophan to niacin. Leucine at a dose exceeding 500 mg/kg/d was observed with hyperammonemia. As such, unofficially, a tolerable upper intake level (UL) for leucine in healthy adult men can be suggested at 500 mg/kg/d or 35 g/d under acute dietary conditions (Elango R, The American Journal of Clinical Nutrition. 2012, 96 (4): 759-67; Rasmussen B, Amino Acids. 2016, 48 (7): 1707-16.)

In some embodiments, the moisture-proof leucine accounts for approximately 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 percent of the total weight of the moisture-proof excipient and the amino acid.

In some embodiments, the moisture-proof excipient is added into the first solution or suspension that contains the amino acid.

In some embodiments, the moisture-proof excipient is firstly dispersed in a dispersion medium. Here, the dispersion medium could be the same dispersion medium as used to form the first solution or suspension. The dispersed moisture-proof excipient is then mixed with the first solution or suspension to form the second solution or suspension.

In some embodiments, the dispersion medium is water.

In some embodiments, after mixing the amino acid and moisture-proof excipient together in the dispersion medium to form a solution or suspension, the solution or suspension is then undergoing a drying process to remove the dispersion medium to obtain a moisture-proof amino acid material. In some embodiments, the drying process includes spray drying, reduced pressure drying, freeze drying, or microwave drying. Those of skill in the art will know, or can readily discern, other drying formats without undue experimentation.

Spray drying is a method of producing a dry powder from a liquid or slurry by rapidly drying with a hot gas. This is the preferred method of drying of many thermally sensitive materials such as foods and pharmaceuticals, or materials which may require extremely consistent, fine, particle size.

Reduced pressure drying, or vacuum drying, is the mass transfer operation in which the moisture present in a substance, usually a wet solid, is removed by means of creating a vacuum. Reduced pressure drying is generally used for the drying of substances which are hygroscopic and heat sensitive, and is based on the principle of creating a vacuum to decrease the chamber pressure below the vapor pressure of the water, causing it to boil.

Freeze Drying is a process in which a completely frozen sample is placed under a vacuum in order to remove water or other solvents from the sample, allowing the ice to change directly from a solid to a vapor without passing through a liquid phase.

Microwave drying is a process that basically works in the same way as when food is heated in a microwave oven. This drying method is based on microwaves penetrating the moist material, where the microwaves are converted into heat and the moisture turns into vapor. It is extensively used to dry food materials due to the high drying rate and uniform drying. Microwave drying can also improve the quality of some food materials. During microwave drying, vapors are generated in the interior of food materials due to volumetric heating. These vapors are forced to move outside due to the pressure gradient. Hence, microwave drying can prevent the shrinkage of food materials. In order to improve the drying efficiency and quality of dried product, microwave drying can be combined with other drying methods.

In some embodiments, the drying process includes two or more of the following drying methods: spray drying, reduced pressure drying, freeze drying, and microwave drying.

In some embodiments, a moisture-proof amino acid material prepared with the methods described herein has reduced moisture attraction. In some other embodiments, a moisture-proof amino acid material prepared with the methods described herein has better anti-caking performance than the raw amino acid. Still, in some embodiments, a moisture-proof amino acid material prepared with the methods described herein is less prone to forming powder holding unity block. Moreover, due to its moisture-proof features, the moisture-proof amino acid material prepared according to methods disclosed herein will cause less weighing or feeding issues during the production process of products containing the amino acid material and will have less effects on the quality and stability of the final products.

Compared to existing methods, the methods disclosed herein do not involve chemical reactions and thus not causing environmental pollution. In addition, the methods disclosed herein are simple and mild, which do not damage the structure and physiological activity of amino acids. The whole process of the methods disclosed herein is green, environmentally friendly, low energy consumption, and is conducive to industrial production.

Some embodiments relate to a composition comprising the moisture-proof amino acid material prepared from any methods disclosed herein.

Some embodiments relate to a nutritional supplement containing the moisture-proof amino acid materials obtained herein. A nutritional supplement is a substance that can increase the levels of protein, energy, calcium, phosphorus and other nutrients, producing a diet more suited to the nutritional needs. A nutritional supplement containing the moisture-proof amino acid materials can increase the levels of protein or energy of a food or food additive.

Unless defined otherwise, all technical and scientific terms used herein have the same meaning as is commonly understood by one of ordinary skill in the art. In the event that there is a plurality of definitions for a term herein, those in this section prevail unless stated otherwise.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements of method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; and use of terms like ‘preferably,’ ‘preferred,’ ‘desired,’ or ‘desirable,’ and words of similar meaning should not be understood as implying that certain features are critical, essential, or even important to the structure of function, but instead as merely intended to highlight alternative or additional features that may or may not be utilized in a particular embodiment. In addition, the term “comprising” is to be interpreted synonymously with the phrases “having at least” or “including at least”. When used in the context of a process, the term “comprising” means that the process includes at least the recited steps, but may include additional steps. When used in the context of a compound, composition or device, the term “comprising” means that the compound, composition or device includes at least the recited features or components, but may also include additional features or components. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

Any titles or subheadings used herein are for organization purposes and should not be used to limit the scope of embodiments disclosed herein.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

### Example 1

This example describes a method of preparing 5% moisture-proof-aminoisobutyric acid material.

The method includes the following steps: 1) prepared a solution of β-aminoisobutyric acid by dissolving 950 g of β-aminoisobutyric acid in 1 L of water; 2) weighed 50 g of the moisture-proof excipient leucine, added it to the aqueous solution in step 1) and mixed well; 3) the water in the solution prepared in step 2) was removed by spray drying to obtain 5% moisture-proof β-aminoisobutyric acid material.

### Example 2

This example describes a method of preparing 8% moisture-proof β-aminoisobutyric acid material.

The method includes the following steps: 1) prepared a solution of ß-aminoisobutyric acid by dissolving 920 g of ß-aminoisobutyric acid in 1 L of water; 2) weighed 80 g of the moisture-proof excipient leucine, added it to the aqueous solution in step 1) and mixed well; 3) the water in the solution prepared in step 2) was removed by spray drying to obtain 8% moisture-proof β-aminoisobutyric acid material.

### Example 3

This example describes a method of preparing 10% moisture-proof β-aminoisobutyric acid material.

The method includes the following steps: 1) prepared a solution of ß-aminoisobutyric acid by dissolving 900 g of ß-aminoisobutyric acid in 1 L of water; 2) weighed 100 g of the moisture-proof excipient leucine, added it to the aqueous solution in step 1) and mixed well; 3) the water in the solution prepared in step 2) was removed by spray drying to obtain 10% moisture-proof β-aminoisobutyric acid material.

### Example 4

This example describes a method of preparing 20% moisture-proof ß-aminoisobutyric acid material.

The method includes the following steps: 1) prepared a solution of ß-aminoisobutyric acid by dissolving 800 g of β-aminoisobutyric acid in 1 L of water; 2) weighed 200 g of the moisture-proof excipient leucine, added it to the aqueous solution in step 1) and mixed well; 3) the water in the solution prepared in step 2) was removed by spray drying to obtain 20% moisture-proof β-aminoisobutyric acid material.

### Example 5

This example describes a method of preparing 5% moisture-proof β-Alanine material.

The method includes the following steps: 1) prepared a solution of β-Alanine by dissolving 950 g of β-Alanine in 1 L. of water; 2) weighed 50 g of the moisture-proof excipient leucine, added it to the aqueous solution in step 1) and mixed well; 3) the water in the solution prepared in step 2) was removed by spray drying to obtain 5% moisture-proof β-Alanine material.

### Example 6

This example describes a method of preparing 8% moisture-proof β-Alanine material.

The method includes the following steps: 1) prepared a solution of β-Alanine by dissolving 920 g of β-Alanine in 1 L of water; 2) weighed 80 g of the moisture-proof excipient leucine, added it to the aqueous solution in step 1) and mixed well; 3) the water in the solution prepared in step 2) was removed by spray drying to obtain 8% moisture-proof β-alanine material.

### Example 7

This example describes a method of preparing 10% moisture-proof β-Alanine material.

The method includes the following steps: 1) prepared a solution of β-Alanine by dissolving 900 g of β-Alanine in 1 L of water; 2) weighed 100 g of the moisture-proof excipient leucine, added it to the aqueous solution in step 1) and mixed well; 3) the water in the solution prepared in step 2) was removed by spray drying to obtain 10% moisture-proof β-Alanine material.

### Example 8

This example describes a method of preparing 20% moisture-proof β-Alanine material.

The method includes the following steps: 1) prepared a solution of β-Alanine by dissolving 800 g of β-Alanine in 1 L of water; 2) weighed 200 g of the moisture-proof excipient leucine, added it to the aqueous solution in step 1) and mixed well; 3) the water in the solution prepared in step 2) was removed by spray drying to obtain 20% moisture-proof β-Alanine material.

### Example 9

This example tests and evaluates the humidification ability of various β-aminoisobutyric acid material or moisture-proof β-Alanine material.

Specific test methods are as follows:
1. Took a dry stoppered glass weighing bottle (outer diameter of 50 mm, height of 15 mm), placed it in an artificial climate chamber (set temperature at 25° C., relative humidity at 80%) one day before the test, and obtained the weight (m₁) of the weighing bottle.
2. Took an appropriate amount of the test sample, laid it flat in the above weighing bottle, the thickness of the test sample was about 1 mm, and obtained the weight of the weighing bottle that contains the test sample (m₂)
3. Left the weighing bottle open in the aforementioned artificial climate chamber.
4. Obtained the weight of the weighing bottle that contains the test sample (m₃) at 5 min, 10 min, 20 min, 30 min, 60 min, 120 min, 180 min, respectively.
5. Calculated the weight gain percentage based on the calculation formula:

$$Weight gain percentage = \frac{{m}_{3} - {m}_{2}}{{m}_{2} - {m}_{1}} \times 100 \%$$

The results of moisture absorption and weight gain of β-aminoisobutyric acid are shown in Table 1 below.

TABLE 1
Results of moisture absorption and weight gain of β-aminoisobutyric acid

| Item | 5 min | 10 min | 20 min | 30 min | 60 min | 120 min | 180 min |
|---|---|---|---|---|---|---|---|
| β-aminoisobutyric acid | 7.8% | 15.9% | 23.7% | 34.6% | 44.8% | 66.8% | 99.6% |
| raw material | | | | | | | |
| 5% moisture-proof β- | 3.2% | 5.8% | 13.2% | 31.2% | 37.5% | 60.5% | 90.7% |
| aminoisobutyric acid | | | | | | | |
| 8% moisture-proof β- | 2.8% | 3.4% | 9.6% | 30.8% | 36.1% | 55.8% | 85.1% |
| aminoisobutyric acid | | | | | | | |
| 10% moisture-proof β- | 1.7% | 2.5% | 7.3% | 22.1% | 29.9% | 42.0% | 78.5% |
| aminoisobutyric acid | | | | | | | |
| 20% moisture-proof β- | 1.1% | 1.8% | 6.1% | 16.4% | 21.9% | 32.5% | 56.4% |
| aminoisobutyric acid | | | | | | | |

The results of moisture absorption and weight gain of β-Alanine are shown in Table 2.

TABLE 2
Results of moisture absorption and weight gain of β-Alanine

| Item | 5 min | 10 min | 20 min | 30 min | 60 min | 120 min | 180 min |
|---|---|---|---|---|---|---|---|
| β-alanine raw material | 4.0% | 8.6% | 11.2% | 14.5% | 26.2% | 55.0% | 71.7% |
| 5% moisture-proof β- | 2.5% | 5.2% | 8.9% | 11.3% | 23.0% | 45.7% | 68.2% |
| alanine | | | | | | | |
| 8% moisture-proof β- | 2.0% | 4.8% | 8.2% | 9.6% | 18.6% | 40.2% | 59.2% |
| alanine | | | | | | | |
| 10% moisture-proof β- | 1.5% | 4.3% | 7.3% | 8.9% | 16.0% | 36.1% | 49.8% |
| alanine | | | | | | | |
| 20% moisture-proof β- | 0.9% | 2.1% | 5.2% | 6.9% | 11.3% | 21.1% | 31.7% |
| alanine | | | | | | | |

From Table 1 and Table 2, it can be seen that the moisture-proof amino acid materials prepared by the disclosed n od has a significant anti-moisture absorption effect under high humidity conditions than the raw materials of amino acids. It can also be seen that the higher the proportion of excipients, the stronger the anti-moisture absorption performance.

1. A method for preparing a moisture-proof amino acid material, comprising the following steps:
1) preparing a first solution or suspension of an amino acid by mixing the amino acid in a dispersion medium,
2) mixing a moisture-proof excipient with the first solution or suspension to form a second solution or suspension, and
3) drying the second solution or suspension to remove the dispersion medium to obtain the moisture-proof amino acid material.

2. The method of claim 1, wherein the amino acid comprises one or more amino acids selected from the group consisting of alanine, aminoisobutyric acid, phenylalanine, tryptophan, methionine, valine, proline, glycine, serine, threonine, cysteine, aspartic acid, lysine, arginine, histidine, and glutamate.

3. The method of claim 1, wherein the amino acid comprises β-aminoisobutyric acid or β-alanine.

4. The method of claim 1, wherein the moisture-proof excipient accounts for 1% to 30% (by weight) of the total weight of the moisture-proof excipient and the amino acid(s).

5. The method of claim 1, wherein the moisture-proof excipient accounts for 5% to 20% of the total weight of the moisture-proof excipient and the amino acid.

6. The method of claim 1, wherein the moisture-proof excipient comprises leucine.

7. The method of claim 1, wherein the moisture-proof excipient is firstly dispersed in a dispersion medium, then mixed with the first solution or suspension to form the second solution or suspension.

8. The method of claim 1, wherein the dispersion medium comprises water.

9. The method of claim 1, wherein the drying of step 3) comprises spray drying, reduced pressure drying, freeze drying, or microwave drying.

10. The method of claim 1, wherein the drying of step 3) comprises spray drying.

11. The method of claim 1, wherein the drying process comprises two or more of the following drying methods: spray drying, reduced pressure drying, freeze drying, and microwave drying.

12. A composition comprising the moisture-proof amino acid material prepared by the method of claim 1.

13. A nutritional supplement comprising the moisture-proof amino acid material prepared by the method of claim 1.
